(19) United States
(12) Patent Application Publication
(10) Pub. No.: US 2009/0013439 A1
(43) Pub. Date: Jan. 15, 2009

(54) Safety glasses

(76) Inventor: Eric Thoman, Rome, PA (US)

Correspondence Address: JAMES RAY & ASSOCIATES, 2640 PICTCAIRN ROAD, MONROEVILLE, PA, 15146, US

(21) Appl. No.: 12/218,164

(22) Filed: Jul. 11, 2008

Related U.S. Application Data
(60) Provisional application No. 60/949,705, filed on Jul. 13, 2007.

(51) Int. Cl. A61F 9/00; A61F 9/02

(52) U.S. Cl. 2/10; 2/435; 2/446; 2/453

(57) ABSTRACT

A pair of safety glasses for use in combination with a hat having a brim is provided. Such pair of safety glasses includes a generally transparent protective member manufactured from a first predetermined material and having a first predetermined size and a first predetermined shape for protecting at least a predetermined portion of a user's face from predetermined detrimental environmental conditions. A securing mechanism has a first portion removably connected to such protective member and a second portion removably connected to such hat brim for securing such protective member to such hat brim. The securing mechanism is manufactured from a second predetermined material and has a second predetermined shape and a second predetermined size.

### CROSS REFERENCE TO RELATED APPLICATION

This patent application is related to and claims priority from U.S. Provisional Patent Application Ser. No. 60/949,705 filed Jul. 13, 2007.

### FIELD OF THE INVENTION

The present invention relates, in general, to glasses and, more particularly, this invention relates to safety glasses and hats.

### BACKGROUND OF THE INVENTION

Prior to the conception and development of the present invention, as is generally well known in the prior art, safety glasses and goggles have been used to protect a user's eyes. However, most goggles and glasses pinch the user's ears or nose, have the tendency to fog up or trap sweat, and irritate such user's skin. Furthermore, such glasses and goggles can become misplaced or lost.

Specifically of interest to the present invention are the following: Holm, U.S. Pat. No. 6,959,989 discloses a system for attaching safety glasses to headwear. Multiple adjustments fit the glasses to the individual user and that allow the user to flip the glasses back and forth between in-use and removed positions. The system includes a base piece that attaches to the interior or underside of the helmet, near the wearer's face at or near the junction of the helmet's bill/brim and dome. A preferred second piece receives a portion of the glasses at its distal end, and, at its proximal end, slideably connects to the base piece. The second piece may be locked at various positions relative to the base piece, to adjust, either incrementally or continuously, the distance of the glasses from the helmet.

Solo, U.S. Pat. No. 5,129,102 discloses a cap provided with removal flip up and down glasses including a mounting member for positioning the flip up and down glasses on an outwardly extending visor of the cap. Attachment members pivotally secure a pair of lenses of the flip up and down glasses to the mounting member so that the glasses can be flipped up to a first position adjacent to the visor and also flipped down to a second position so that the lenses are disposed at a proper eye level for a person wearing the cap. Separable fasteners are disposed between the visor and the mounting member for removably securing the visor and the mounting member together. One portion of the fasteners is secured on an underside of the visor and another mating portion of the fasteners is secured to an upper surface of the mounting member.

Garrett, U.S. Pat. No. 5,347,655 discloses a sun visor with attached sunglasses rotatable incrementally between an up position, a down position and several intermediate positions. The frame of the sunglasses has a pair of posts extending laterally through holes in members dependent from the visor. The holes both have scalloped edges and the posts have a prismatic shape so that the scallops will engage the sides of the prismatic post and resist its rotation but not prevent rotation if sufficient force is applied to deform the scallops. The ends of the posts are conical and slotted to facilitate insertion of the posts through the holes in the dependent members and prevent removal because the diameter of the base of the cone is larger than the diameter of the hole.

Bates, U.S. Pat. No. 4,432,100 discloses a transparent safety visor which may be retrofitted to protective hardhat helmets. Such helmets typically consist of an inverted bowl defining a number of key receptor sites at intervals around its interior circumference, and a liner cap including a central crown and a number of struts radiating there from and terminating in keys adapted to be releasably locked to the receptor sites to maintain the liner cap and bowl in a fixed relationship. The visor is made of a flexible, transparent, generally rectangular plastic sheet, with at least a pair of slots in the sheet through which are inserted a corresponding number of liner cap keys prior to placement thereof in the receptor sites. The lower edge of the visor extends below the edge of the helmet to provide a gripping surface for user adjustment of the position of the visor, and the upper edge of the visor slides between the liner cap and the bowl when adjusted.

### SUMMARY OF THE INVENTION

The present invention provides a pair of safety glasses for use in combination with a hat having a brim. Such pair of safety glasses includes a generally transparent protective member manufactured from a first predetermined material and having a first predetermined size and a first predetermined shape for protecting at least a predetermined portion of a user's face from predetermined detrimental environmental conditions. A securing mechanism has a first portion removably connected to such protective member and a second portion removably connected to such hat brim for securing such protective member to such hat brim. The securing mechanism is manufactured from a second predetermined material and has a second predetermined shape and a second predetermined size.

### OBJECTS OF THE INVENTION

It is, therefore, one of the primary objects of the present invention to provide a pair of safety glasses for attachment to a hat having a brim.

Another object of the present invention is to provide a pair of safety glasses for attachment to a hat such that the glasses protect a user's eyes and face without touching such user's face.

Still another object of the present invention is to provide a pair of safety glasses which will not fog up, trap sweat, or irritate a user's skin.

Yet another object of the present invention is to provide a pair of safety glasses attachable to a hat having a brim such that such glasses are moveable between a generally horizontal position and a generally vertical position.

Still another object of the present invention is to provide an easy way for a user to keep track of his or her safety glasses without fear of loss, theft, and accidental destruction.

An additional object of the present invention is to provide a way for a user to wear safety glasses comfortably.

In addition to the various objects and advantages of the present invention described with some degree of specificity above it should be obvious that additional objects and advantages of the present invention will become more readily apparent to those persons who are skilled in the relevant art from the following more detailed description of the invention, particularly, when such description is taken in conjunction with the attached drawing figures and with the appended claims.

### BRIEF DESCRIPTION OF THE DRAWINGS

FIG. 1 is a partial perspective view of the invention according to one embodiment of the invention.

FIG. 2 is a partial perspective side view of the invention according to one embodiment of the invention.

FIG. 3 is a partial perspective view of the invention according to one embodiment of the invention.

FIG. 4 is a partial perspective view of the invention according to one embodiment of the invention.

### BRIEF DESCRIPTION OF A PRESENTLY PREFERRED AND VARIOUS ALTERNATIVE EMBODIMENTS OF THE INVENTION

Prior to proceeding to the more detailed description of the present invention it should be noted that, for the sake of clarity and understanding, identical components which have identical functions have been identified with identical reference numerals throughout the several views illustrated in the drawing figures.

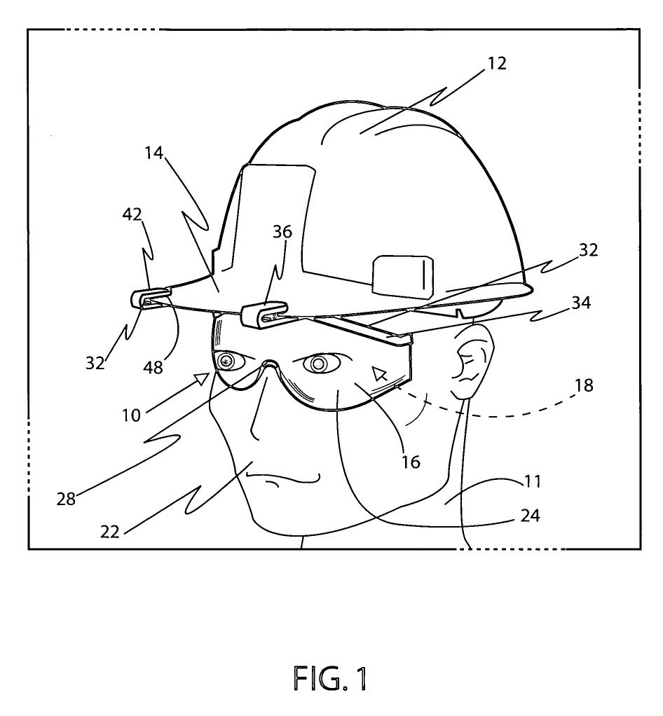

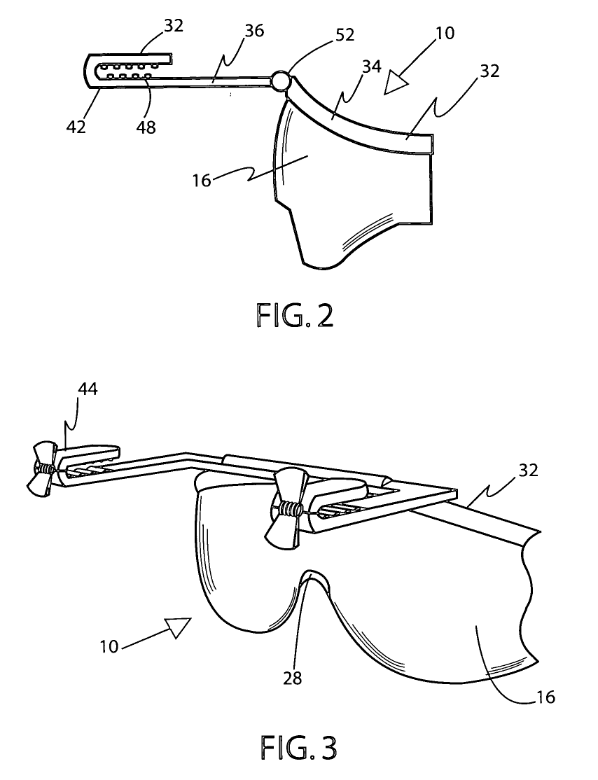

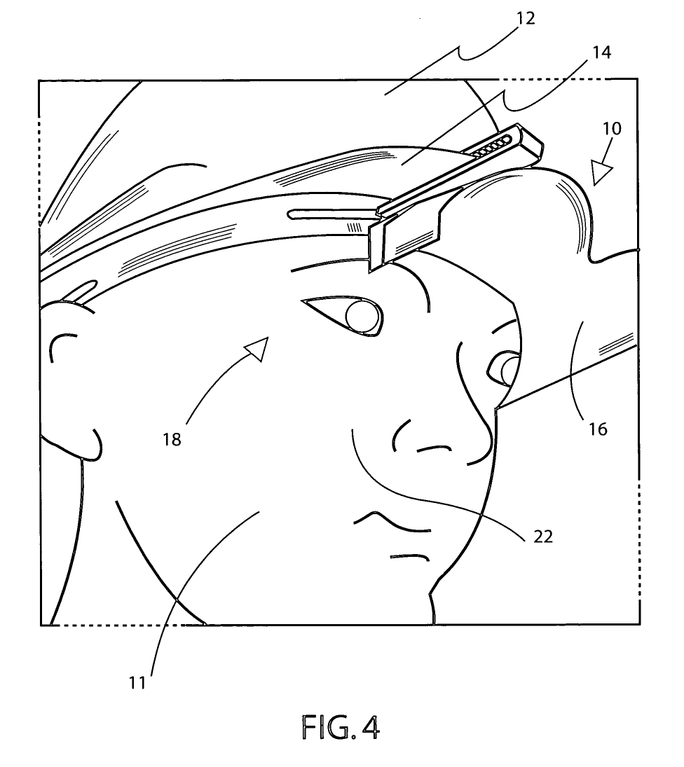

Reference is now made, more particularly, to FIGS. 1-4.

A pair of safety glasses, generally designated 10, for use in combination with a hat 12 having a brim 14, is provided.

Such pair of safety glasses 10 includes a generally transparent protective member 16 manufactured from a first predetermined material and having a first predetermined size and a first predetermined shape for protecting at least a predetermined portion generally designated 18 of a user's 11 face 22 from predetermined detrimental environmental conditions.

It is presently preferred that such predetermined detrimental environmental conditions include at least one of dirt, dust, debris, sand, splinters, airborne pollutants, sun, rain, snow, wind, weather elements, metal shards, gases, steam, chemicals, liquid splatters, liquid spray, insects, and a combination thereof. However, the environmental conditions are not meant to be limited as such and may include any conditions within a user's environment which may be detrimental.

It is presently preferred that the first predetermined material is plastic.

It is presently preferred that such protective member 16 includes UV protection.

It is further presently preferred that such protective member 16 includes tinting.

It is presently preferred that such protective member 16 is fog resistant.

It is presently preferred that such protective member 16 includes removable lenses 24.

It is presently preferred that such first predetermined shape is curvilinear to align with features of such user's face 22.

It is presently preferred that such protective member 16 is about 2.5 inches high, about 6.0 inches long, and about 4.0 inches wide.

It is presently preferred that such protective member 16 includes a nosepiece 28 formed therein.

A securing means 32 has a first portion 34 removably connected to such protective member 16 and a second portion 36 removably connected to such hat brim 14 for securing such protective member 16 to such hat brim 14. The securing means 32 is manufactured from a second predetermined material and has a second predetermined shape and a second predetermined size.

It is presently preferred that the second predetermined material is at least one of metal, plastic, and a combination thereof. It is presently most preferred that such second predetermined material is plastic.

It is presently preferred that such securing means 32 is at least one of a clamp type securing means 42, a clip type securing means 44 and a combination thereof. It is presently most preferred that such securing means 32 is a clamp type securing means 42. It is further presently preferred that such clamp type securing means 42 includes ridges 48 disposed thereon at a predetermined location for gripping such hat brim 14.

It is presently preferred that such securing means 32 further includes at least one pivot member 52 connected to each of such first portion 34 and such second portion 36 thereof for enabling such protective member 16 to pivot between a generally vertical position in which such protective member 16 shields such at least such predetermined portion 18 of such user's face 22 and a generally horizontal position in which such protective member 16 is raised substantially above such user's line of vision.

While a presently preferred and various alternative embodiments of the present invention have been described in sufficient detail above to enable a person skilled in the relevant art to make and use the same it should be obvious that various other adaptations and modifications can be envisioned by those persons skilled in such art without departing from either the spirit of the invention or the scope of the appended claims.

I claim:

1. A pair of safety glasses for use in combination with a hat having a brim comprising:
a. a generally transparent protective member manufactured from a first predetermined material and having a first predetermined size and a first predetermined shape for protecting at least a predetermined portion of a user's face from predetermined detrimental environmental conditions; and
b. a securing means having a first portion removably connected to said protective member and a second portion removably connected to such hat brim for securing said protective member to such hat brim, said securing means being manufactured from a second predetermined material and having a second predetermined shape and a second predetermined size.

2. A pair of safety glasses for use in combination with a hat having a brim according to claim 1 wherein said first predetermined material is plastic.

3. A pair of safety glasses for use in combination with a hat having a brim according to claim 1 wherein said protective member includes UV protection.

4. A pair of safety glasses for use in combination with a hat having a brim according to claim 1 wherein said protective member includes tinting.

5. A pair of safety glasses for use in combination with a hat having a brim according to claim 1 wherein said securing means is at least one of a clamp type securing means, a clip type securing means and a combination thereof.

6. A pair of safety glasses for use in combination with a hat having a brim according to claim 5 wherein said securing means is a clamp type securing means.

7. A pair of safety glasses for use in combination with a hat having a brim according to claim 6 wherein said clamp type securing means includes ridges disposed thereon at a predetermined location for gripping such hat brim.

8. A pair of safety glasses for use in combination with a hat having a brim according to claim 1 wherein said second predetermined material is at least one of metal, plastic, and a combination thereof.

9. A pair of safety glasses for use in combination with a hat having a brim according to claim 8 wherein said second predetermined material is plastic.

10. A pair of safety glasses for use in combination with a hat having a brim according to claim 1 wherein said securing means further includes at least one pivot member connected to each of said first portion and said second portion thereof for enabling said protective member to pivot between a generally vertical position in which said protective member shields such at least such predetermined portion of such user's face and a generally horizontal position in which such protective member is raised substantially above such user's line of vision.

11. A pair of safety glasses for use in combination with a hat having a brim according to claim 1 wherein said protective member is fog resistant.

12. A pair of safety glasses for use in combination with a hat having a brim according to claim 1 wherein said protective member includes removable lenses.

13. A pair of safety glasses for use in combination with a hat having a brim according to claim 1 wherein said first predetermined shape is curvilinear to align with features of such user's face.

14. A pair of safety glasses for use in combination with a hat having a brim according to claim 1 wherein said predetermined detrimental environmental conditions include at least one of dirt, dust, debris, sand, splinters, airborne pollutants, sun, rain, snow, wind, weather elements, metal shards, gases, steam, chemicals, liquid splatters, liquid spray, insects, and a combination thereof.

15. A pair of safety glasses for use in combination with a hat having a brim according to claim 1 wherein said protective member includes a nosepiece formed therein.
